(19) United States
(12) Patent Application Publication
(10) Pub. No.: US 2005/0133026 A1
(43) Pub. Date: Jun. 23, 2005

(54) Device for the treatment of snoring and obstructive sleep apnea

(76) Inventors: Katie Seleznev, West Hartford, CT (US); Stanislav Vitruk, West Hartford, CT (US)

Correspondence Address: Katie Seleznev, 149 Brewster Road, West Hartford, CT, 06117, US

(21) Appl. No.: 10/746,707

(22) Filed: Dec. 23, 2003

(51) Int. Cl. A62B 7/00; A61M 15/00

(52) U.S. Cl. 128/200.24; 128/848

(57) ABSTRACT

The present invention is the universal unmedicamental system to treat the snore and obstructive sleep apnea and recover of the normal breathing during sleep. The invention includes the device, which ensures the fixation of the tongue in a putting forward beyond the mouth position. As a result, the tongue's pharyngeal part pushes forward in the mouth, releasing besides the throat and excluding the tongue from a system of obstruction to the air's passing through the throat. This increases the throat's capacity, depriving the other throat's structures of the possibility to provoke the partial or complete obstruction to the air's passing through the throat during the physiological process of breathing in the time of the sleep.

### BACKGROUND OF THE INVENTION

1. Technical Field

The present invention is the universal unmedicamental system to treat the snore and obstructive sleep apnea and to lead to the recovery of normal breathing during sleep.

2. Background Art

In the time of normal breathing, the air passes in the throat through the flexible structures (the soft palate, uvula, tonsils, and tongue) easily, without noise. While awake, muscles around each structure tighten to hold the structure in place so it doesn't block the air's passage. During sleep, these muscles relax, but the passage to the throat still stays clear enough for air to flow freely into and out of the lungs.

During sleep, some people have greater relaxation of structures' muscles and as a result occurs the considerable loss of their elasticity. The tongue shortens and thickens, its protruding root sticks in the throat, and together with the uvula, soft and hard palates blockade the way of the air's passing. The passage of the air from a nose or mouth through this blocked canal causes the throat's vibration and the appearance of the snoring sound. If the tongue is sticking inside the throat, the soft palate's structures are pressed with the tongue to the throat's back wall, causing the complete blockade of the air's passing into the lungs. This condition is called sleep apnea.

Snore and sleep apnea happen to men and women of different ages. But with its increase, the quantity of people suffering from this deficiency is growing. At first, there is a small block of air and the sleep snore is short. Little by little, the disease develops, the block of air grows, and sleep snoring becomes loud and irritating to the surroundings, interchanging with frequent obstructive sleep apnea.

Although a person doesn't hear his or her own snore, it causes harm to his health. Snore and sleep apnea disturb the breath and cause the hipoxia of the brain, and do the sleep uneasiness and irregularity. After the uneasy night, a person awakes with a headache, irritability, and sleeplessness, which may cause the person to possible fall asleep behind a car's wheel. Disturbing dreams are accompanied with stress, which lead to increased blood pressure, and possible heart attack and/or stroke.

At the present time, for the treatment of snoring and sleep apnea, there are devices used which push forward the lower jaw, but they do not give due effect. The appliance Continuous Positive Airway Pressure (CPAP) has significant shortcomings too, and they restrict its employment. The main shortcoming of CPAP is the uninterrupted blow under pressure with the generator the air's flow through the nose into the lungs. This causes the soft palate's undesirable putting down in the throat to the tongue's pharyngeal part, the difficulty of the exhalation, and is the contraindication for people with existing respiratory failure, decompensated cardiac failure, diseases of the nose, ear, etc. CPAP is bulky, works from the electrical transmission network, does not function when the patient's mouth is open, and prevents a person to sleep. The price of CPAP and tests before its prescription are often beyond the means for people. All this causes the necessity in new inventions to treat the snore and the obstructive sleep apnea.

The purpose for the present invention is the treatment of the snore and obstructive sleep apnea for sick persons of any age, regardless of their diseases and without any influence on the physiological act of the breath during the inhalation and the exhalation; the rendering with the appliance of the medical effect without any additional factors of the influence; the simplicity and the compactness of the device, enabling to use it in any person's living conditions; the absence of contraindications for its employment; the recommendation of it without any special tests.

This aim was realized by the expulsion of the tongue—the main object between the obstructive structures of the throat—from the process of the obstruction of the air's passing through the throat. Tongue's moving forward from the mouth, with the fixation of the tongue with the aid of the proposed device is new in the decision of practical task of the treatment of snore and the obstructive sleep apnea. In the time of the tongue's moving in the front position, its pharyngeal part shirts in the mouth. The throat's cavity extends, and it gets impossible for the obstruction of the air's passing into the lungs and in the opposite direction. Moving forward of the tongue in the front part of the mouth in the time of sleep does not disturb the physiological breath of both types (through the nose and the mouth), regardless of a person's age and attendant diseases.

### SUMMARY OF THE INVENTION

The present invention is a universal system to treat the sleep snore and the obstructive sleep apnea, and thanks to this provide normal breathing during sleep. Since the tongue plays the decisive role in the obstruction for air's passing through the throat, just the tongue is chosen for the decision of practical task of the removal of this obstruction. With this purpose, the tongue is moved forward and fixed with a special device. One end of this fixation is fastened through an aperture in the oral part of the tongue on the level of a front third of the tongue's bridle and the second end is fastened to the device, put against the chin. The device is fixed from above with the wedge-shaped ledges, which are put on the side teeth of the lower jaw, from below with the semicircular strip situated outside from the lower jaw, and from the sides with strips covering the person's cheeks. The final fixation of the device is realized with the strip being put on the head. The wedge-shaped ledges put on each side of the teeth of the lower jaw also protect the tongue from the bite of the teeth of the upper jaw.

The device put on the lower jaw doesn't prevent-the opening and closing of the mouth. The tongue's front part jutting out the bound of the anterior teeth and the fixation of the tongue are covered with the device's lid.

In the time of the tongue's moving forward, its pharyngeal part advances from the throat in the mouth and as a result the throat's cavity increases, and becomes free for the air's passage. Thanks to an absence of the tongue's pharyngeal part in the throat, other structures become incapable to provoke an obstruction of the air's passing, and as a result, the inhalation and the exhalation become almost noiseless.

The device's design and way to use are simple and haven't any unfavorable influence or risk upon a person's health. Therefore, before the using, it is not required to perform any special tests in a sleep clinic; there is enough of a doctor's examination and recommendation.

Before sleep, the chin body of the device is put to the chin and fixed with straps. Then the tongue sticks out and into the small aperture on its end there is inserted the fixation's hooked front part. The fixation's other end fastens to the device's chin body, after this the tongue with the fixation is covered with a lid, and the lips close spontaneously the mouth.

When the device is installed, the breath remains physiological, the inhalation and exhalation free as with nasal type of the breath as with mouth type. During the nasal type of breath, the tongue's fixation doesn't prevent the closing of the mouth. In the case of some nose diseases (polypuses, partition's curvature, cold, etc.), which make difficulty for the breathing through the nose, the sick person can breathe with the open mouth or use for the full compensation simultaneously both types of breath.

The fastened to the chin device and the tongue's partial moving out the bound of the anterior teeth doesn't prevent to change the body's position and swallow during the sleep. The device is portable, doesn't present any difficulties in the time of its transportation, and can be used at any conditions of a person's life.

### BRIEF DESCRIPTION OF THE DRAWINGS

FIG. 1 is the sagittal section through the nose, mouth and throat. The position of the structures in the throat with the tongue's moving forward from the mouth, and the air's flow in the time of nasal breath.

FIG. 2 is the sagittal section through the nose, mouth and throat. The position of the structures in the throat with the tongue's moving forward from the mouth, and the air's flow in the time of the simultaneous nasal and through the mouth breathing.

FIG. 3 is the sagittal section through the nose, mouth and throat. The position of the tongue and the structures in the throat in the time of the partly blocked air's flow during the snore.

FIG. 4 is the sagittal section through the nose, mouth and throat. The position of the tongue and the structures in the throat in the time of the completely blocked air's flow during the sleep apnea.

FIG. 5 is the sagittal section through the nose, mouth and throat. The position of the tongue, structures in the throat, and the free air's flow in the time of the nasal breathing during the normal sleep.

FIG. 6 is the sagittal section through the nose, mouth and throat. The position of the tongue, structures in the throat, and the free air's flow in the time of the simultaneous nasal and through the mouth breathing during the normal sleep.

FIG. 7 is the general view of the device.

FIG. 8 is the exterior view from the side and from above of the chin body.

FIG. 9 is the exterior view from the side and from below of the chin body.

FIG. 10 is the exterior view of the strip under the lower jaw.

FIG. 11 is the exterior view of the lid.

FIG. 12 is the exterior view of the head strap.

FIG. 13 is the exterior view of the tongue's fixation.

FIG. 14 is the exterior view of the position of the hole in the tongue and the fixation: A—view from above, B—view from below.

FIG. 15 is the sagittal section through the nose, mouth, throat, and the device fastened to the chin and head, and the free air's flow during the sleep.

FIG. 16 is the general view of the device fastened to the chin and head.

### DETAILED DESCRIPTION OF THE PREFERRED EMBODIMENTS

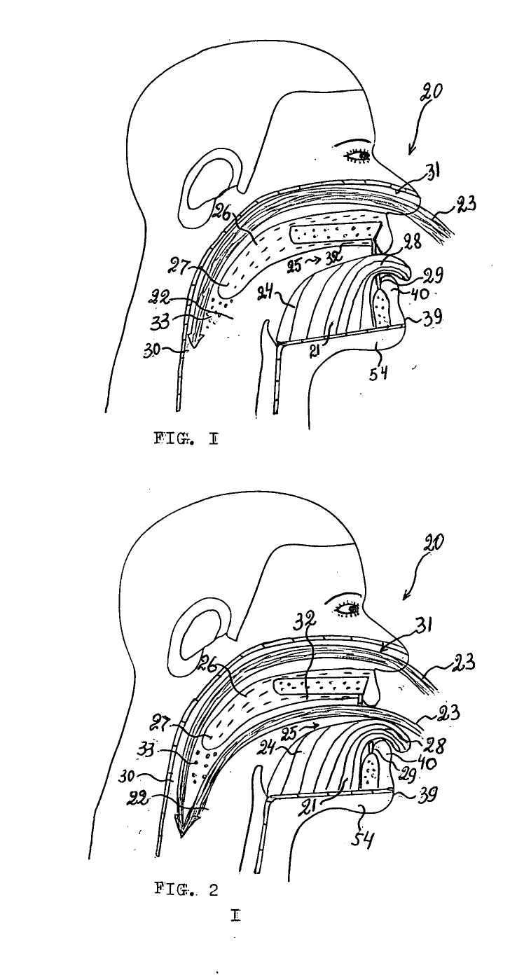

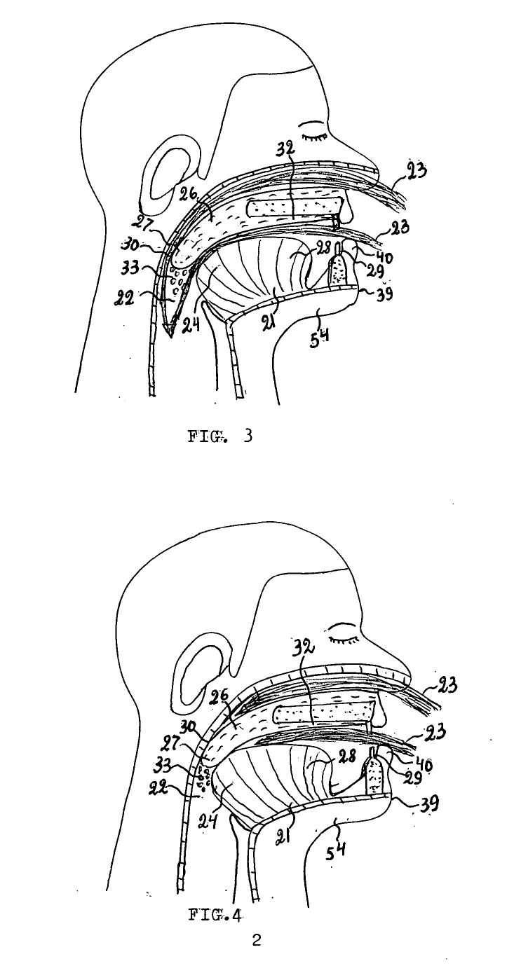

As is shown in the drawings, the present invention 20 to treat the snore and the sleep apnea includes the tongue 21 as the main obstructive structure of the throat 22 in the time of the passing of the air's flow 23 through the throat in the lungs (FIG. 1, 15). Thanks to the fixation of the tongue 21 after its moving in the front position, the tongue's pharyngeal part 24 shifts into the mouth 25, and the tongue excludes from the obstructive process. This deprives in its turn the soft palate's 26 structures and the uvula 27 of the possibility to provoke the partial (FIG. 3) or the complete (FIG. 4) obstruction of the air's passing through the throat into the lungs.

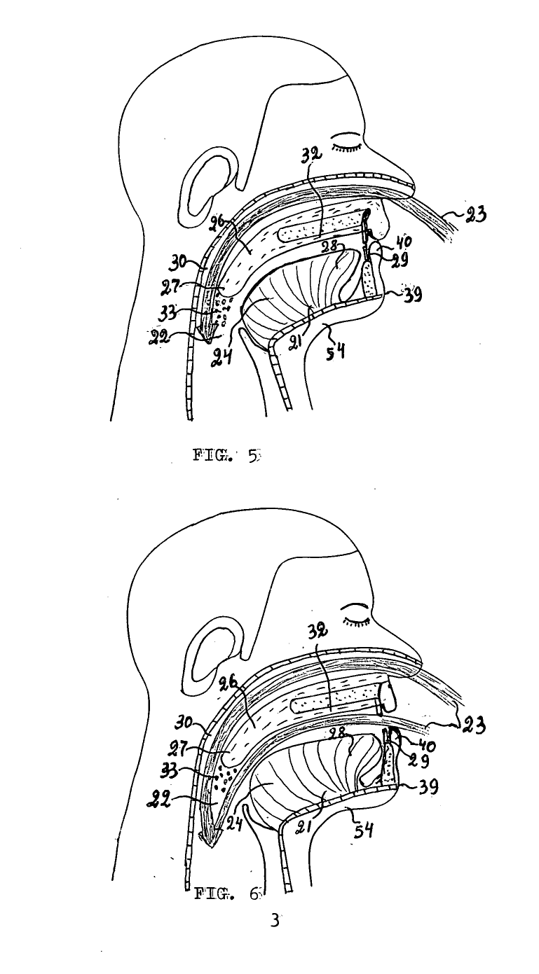

FIG. 3 and FIG. 4 illustrate that in the sleeping time, when the muscles of the soft palate 26 and the tongue 21 relax more normal, they cause the obstruction to the air's passing through the throat. Besides the tongue is main participant in this process. In comparison with normal (FIG. 5), the tongue becomes shorter and thicker, its oral part 28 draws away from the front teeth 29 inside the mouth 25, its pharyngeal part 24 thickens and moves into the throat 22, presses the soft palate's structures 26 to the throat's back side 30, and as a result occurs the complete obstruction of air's passing through the throat, and then comes the sleep apnea (FIG. 4).

Therefore, to decide a practical task of the treatment of the snore and the obstructive sleep apnea, the present invention regards as the main object of the research the tongue. It remains fixed during sleep in the pushed forward from the mouth position (FIG. 1, 15). In this tongue's position (FIG. 1 in the time of the nasal breath, and FIG. 2 during the simultaneous nasal and through the mouth breathing), air's flow 23 goes like during the normal breath (FIG. 5, 6), that is to say free pass by structures of the nose 31 and the mouth 25 (the hard palate 32, the tongue 21), and then pass by structures of the soft palate 26, the uvula 27, the tonsils 33 through the trachea and the bronchia into the lungs and back.

In the time of the tongue's moving forward position (FIG. 1, 15) in comparison with the norm (FIG. 5), the tongue's pharyngeal part 24 shifts from the throat 22 into the mouth 25. Thanks to this, the throat extends in the time of sleep, the tongue excludes from the link of the throat's obstructive structures, occurs the rupture in the obstructive mechanism (FIG. 3, 4).

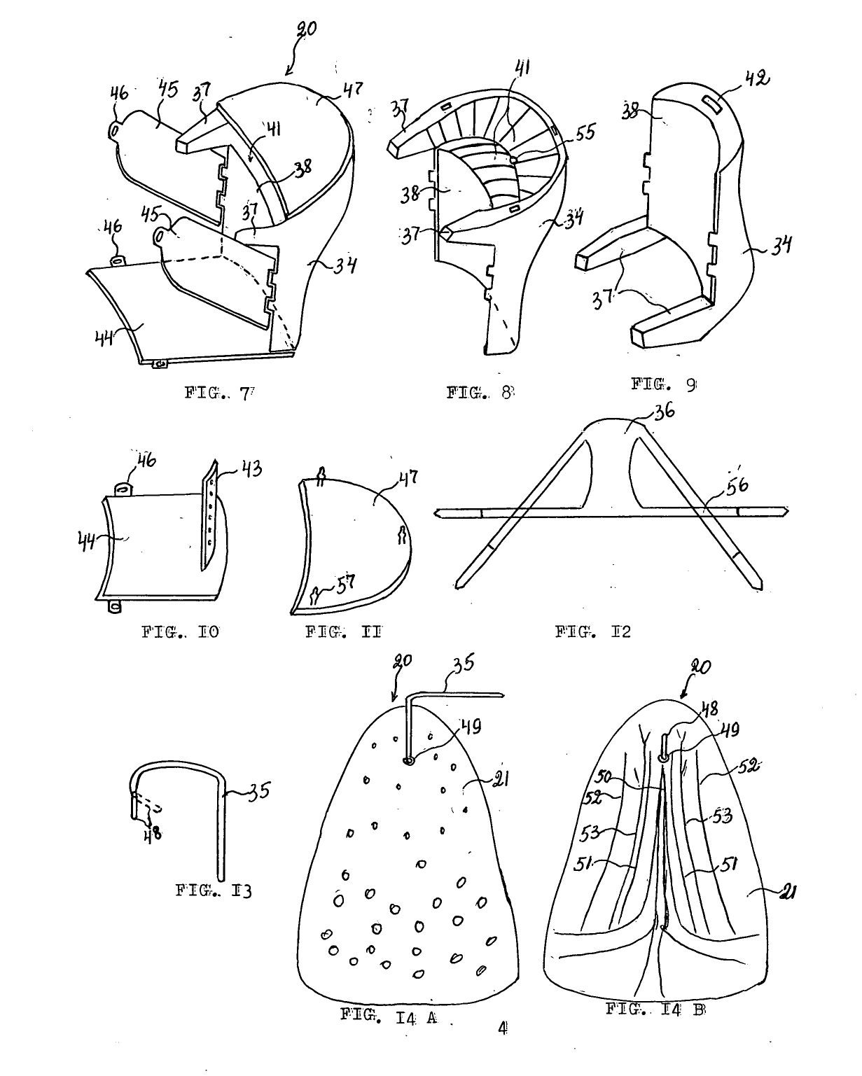

The present invention includes its exterior part (FIG. 7), which consists from the device's chin body 34, the fixation 35 of the tongue (FIG. 13), and the head strap 36 (FIG. 12). The chin body has from above two wedge-shaped ledges 37 (FIG. 8), the recess with U-shaped configuration 41 for the tongue 21 (FIG. 7, 8), the hollow 38 for a chin 39 and lower lip 40 (FIG. 15), and the rectangular aperture 42 (FIG. 9) for inserting of the plug 43 to fasten the mobile strip 44 under the lower jaw 54 (FIG. 10). On sides to the chin body, there are two strips 45 adjoining to people's cheeks with hinges 46 (FIG. 7, 16), and the lid 47 from above (FIG. 7, 11), fastened to the chin body with compressing plugs 57. To the hinges 46 on the strips 45 and 44 adjoining correspondingly to the cheeks and to the lower jaw, is fastened to the head strap 36 (FIG. 12, 16). The tongue's fixation 35 (FIG. 13) is the concave strip with hooked ends. The end inserting into the small hole 49 in the tongue has the mobile tip 48.

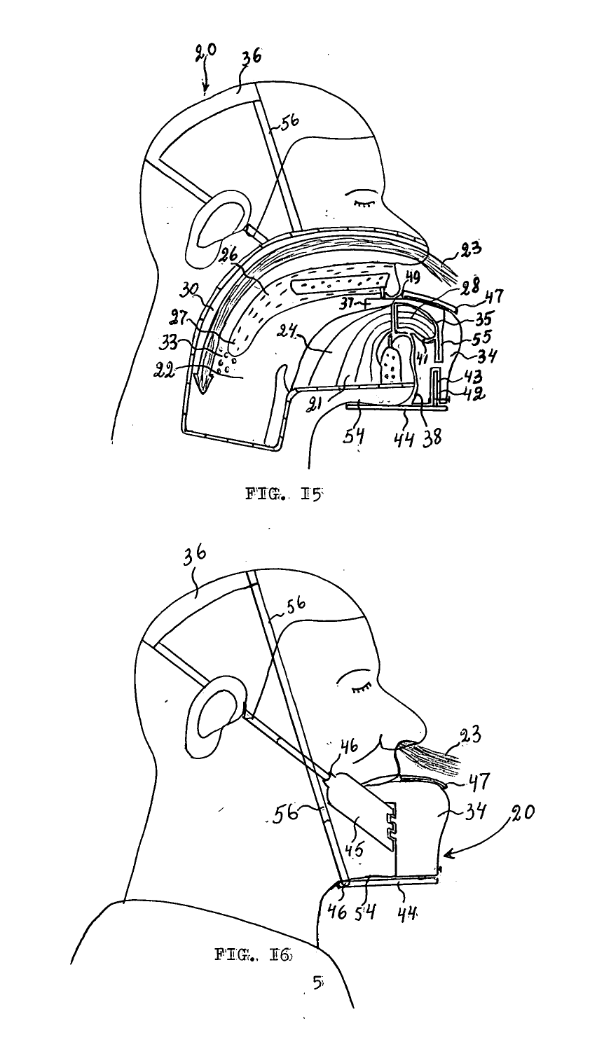

The most effective and reliable for the fixation is the hole 49, made in the oral part 28 of the tongue (FIG. 14 A, 14 B) on its frenulum's end 50 in the distance of the deep lingual artery 51, deep lingual vein 52, and lingual nerve 53.

The wedge-shaped ledges 37 (FIG. 15) settle down on the lower side of the teeth 29. They keep the device's chin body from above and protect the tongue from the bite with teeth. The strip 44 is adjusted tightly under the lower jaw 54 and fastens to the chin body 34, keeping it from below. The tongue 21 moves forward in the hollow 41. From above of the tongue is situated the fixation 35. With one end it grasps the tongue through its hole 49, and with the other end fastens the tongue to the chin body 34 through its aperture 55. A lid 47 covers the tongue from above and preserves it from the dryness during the sleep. The chin body is fixed from the right and left with strips 45 adjoining to the cheeks of a sick person (FIG. 16).

The device's situation on the chin 39 doesn't restrict a person's living conditions, and possibilities to change a body's position during sleep. The breathing and the swallowing are not disturbed. A person can breathe through the nose and through the mouth or both types of breathing simultaneously.

The installation of the device proceeds as follows (FIG. 15, 16). The chin body 34 is put on the chin 39 so that the wedge-shaped ledges 37 sit down on the lower side teeth 29 (in case of their absence—on the gums), and the strip 44 under the lower jaw 54. After this, the strap 36 is put on the head over the ears, and one pair of bridles 56 are fastened to the hinges 46 on the strips 45 adjoining to the cheeks, another pair—to the hinges on the strip 44 under the jaw, and are put on the head in front of the ears. Then, on the moved forward tongue through the hole 49, the fixation 35 is inserted, and the other fixation's end fastens with the chin body. After this, the tongue and the fixation shield with the device's lid 47.

An examination of the device on the person suffering from noisy sleep snore and obstructive sleep apnea was demonstrated with the positive result of the treatment. The device didn't prevent to a process of the people's sleep, and didn't make any inconvenience in the time of changing of the body's positions. The breathing was kept even, noiseless, and the sleep was deep and quiet.

1. The present invention The Device for the Treatment of snoring and Obstructive Sleep Apnea includes:
the tongue as a main structure participating in the obstruction to the air's passing through the throat into the lungs;
the fixation of the tongue with pushed forward its pharyngeal part from the throat in the mouth, and this excludes the tongue's participation in the system of obstruction, depriving thereby the throat's other structures of the possibility to provoke the partial or complete obstruction to the air's passing through the throat into the lungs;
the hole in the tongue's oral part intended for its fixation in a position moved forward beyond of the mouth to the device.

2. The Device for the Treatment of Snoring and Obstructive Sleep Apnea includes:
the hooked fixation with two ends, one of them inserts into the hole in the tongue, and the other fixes the tongue to the device's chin body.

3. The present invention, as defined in claim 1, wherein said about the device, includes:
the chin body with two wedge-shaped ledges which are put into the mouth, keep it in a slightly open condition and protect the tongue's front part from the bite with teeth;
the recess with the U-shaped configuration for the front part of the tongue;
the lid covering the tongue from above when it is in the U-shaped recess;
the mobile semicircular strip for the close adjustment of the chin body to the lower jaw;
left and right strips adjoining the chin body to the person's cheeks;
the strap with two pairs of bridles fastened the device to the head of a sick person.
